(19) United States
(12) Patent Application Publication
(10) Pub. No.: US 2024/0398259 A1
(43) Pub. Date: Dec. 5, 2024

(54) CARDIAC SIGNAL BASED BIOMEDTRIC IDENTIFICATION

(71) Applicant: NETEERA TECHNOLOGIES LTD., Jerusalem (IL)

(72) Inventors: Dana Shavit, Mevasseret Zion (IL); Reinier Doelman, Jerusalem (IL); Ehud Fishler, Shoham (IL)

(21) Appl. No.: 18/699,510

(22) PCT Filed: Oct. 19, 2022
(86) PCT No.: PCT/IL2022/051106

§ 371 Date: Apr. 8, 2024

Related U.S. Application Data
(60) Provisional application No. 63/270,065, filed on Oct. 21, 2021.

(51) Int. Cl. A61B 5/11; A61B 5/00; A61B 5/0507

(52) U.S. Cl. CPC A61B 5/1102; A61B 5/0507; A61B 5/725; A61B 5/7264

(57) ABSTRACT

Method and system for biometric identification. A cardiac signal, such as a ballistocardiogram signal, obtained from a reference subject is segmented into heartbeat segments over selected time duration. Cardiac signal may be obtained using remote non-invasive millimeter-wave radar detector. Linear mapping is applied to each heartbeat segment to produce a respective heartbeat frequency encoding, which is assigned an identification label relating to reference subject. Machine learning process is applied to a collection of heartbeat frequency encodings during a modelling stage to generate a model for subject classification. Model is applied to input heartbeat frequency encoding during an identification stage, to classify input heartbeat frequency encoding as belonging to a reference subject if a matching classification is obtained or to determine that the input heartbeat frequency encoding belongs to a non-reference subject if no matching classification is obtained. Subject identification may be utilized for healthcare monitoring applications.

### FIELD OF THE INVENTION

The present invention generally relates to the fields of biometric identification and classification, temporal signal processing, and machine learning analyses.

### BACKGROUND OF THE INVENTION

Biometric identifiers are biological attributes that are distinct and measurable and can be utilized to describe, identify and categorize an individual person. A biometric identifier may relate to a unique physiological characteristic, such as a fingerprint, ocular features (e.g., iris recognition), facial features, or hand/palm features. One highly reliable form of physiological identification is DNA profiling, which involves analyzing unique variations in DNA sequencing, primarily sequences known as short tandem repeats, for matching DNA samples obtained from bodily fluids. DNA profiling is commonly used nowadays in various applications, ranging from criminal investigations to parentage testing. Biometric identifiers may also be based on behavioral characteristics, examples of which include: signature recognition, voice recognition, and gait analysis.

There have been recent developments in biometric identification based on electrocardiography, which measures the electrical activity of the heart. However, this requires specialized medical equipment, including an electrocardiograph or electrocardiogram (ECG) machine with multiple electrodes that must be placed directly on the patient body (usually the chest or limbs) for obtaining an ECG signal. A qualified practitioner or clinician is generally required to implement proper positioning of the electrodes and to operate the ECG machine. Information relating to the cardiac cycle may also be derived from photoplethysmography (PPG) measurements, which uses optical measurement techniques to monitor volumetric variations in blood circulation. PPG measurements are obtained by illuminating the skin with a light source and then measuring the changes in reflection or absorption of the light with a photodetector, such as by means of a pulse oximeter. Yet such devices usually require components to be in physical contact with a body part of the subject, such as being attached to a finger. There also exist wearable devices, such as smartwatches or chest straps, which incorporate smaller sensors configured to obtain heart rate or cardiac cycle information. However, these wearable heart monitoring devices are often cumbersome and prone to malfunctions and inaccuracies. Moreover, these devices must operate under adequate lighting conditions and must have a direct line-of-sight with clear visibility to the measured skin region. Thus, they cannot function under low light or poor visibility conditions, or through obstructions or occlusions, such as clothing worn by the subject.

Publications describing biometric identification with heartbeat signals include: Paiva, J. S., Dias, D., & Cunha, J. (2017). Beat-ID: Towards a computationally low-cost single heartbeat biometric identity check system based on electrocardiogram wave morphology. PloS one, 12(7), e0180942; Odinaka, I., Lai, P., Kaplan, A. D., O'Sullivan, J., Sirevaag, E., & Rohrbaugh, J. (2012). ECG Biometric Recognition: A Comparative Analysis. IEEE Transactions on Information Forensics and Security, 7, 1812-1824; Calleja, A., Peris-Lopez, P., Tapiador, J. E. Electrical Heart Signals can be Monitored from the Moon: Security Implications for IPI-Based Protocols. In Information Security Theory and Practice, Springer International Publishing: Cham, Switzerland, 2015, pp. 36-51; and Wang, W., Stuijk, S., De Haan, G. Unsupervised subject detection via remote PPG. IEEE Trans. Biomed. Eng. 2015, 62, 2629-2637.

### SUMMARY OF THE INVENTION

In accordance with one aspect of the present invention, there is thus provided a method for biometric identification. The method includes the procedures of obtaining a cardiac signal from at least one reference subject, and segmenting the obtained cardiac signal into a plurality of heartbeat segments over a selected time duration. The method further includes the procedures of applying at least one linear mapping to each of the heartbeat segments to produce a respective heartbeat frequency encoding, and assigning each heartbeat frequency encoding an identification label relating to the reference subject. The method further includes the procedure of applying at least one machine learning process to a collection of heartbeat frequency encodings during a modelling stage, to generate a model for subject classification. The method further includes the procedure of applying the model on an input heartbeat frequency encoding during an identification stage, to classify the input heartbeat frequency encoding as belonging to a reference subject if a matching classification is obtained or to determine that the input heartbeat frequency encoding belongs to a non-reference subject if no matching classification is obtained. The cardiac signal may be a ballistocardiograph (BCG) signal. The cardiac signal may be obtained using contactless means. Segmenting the obtained cardiac signal may be performed by applying at least one process of: peak detection; zero-crossing detection; RR interval detection; and/or interbeat (IBI) interval detection. Applying a linear mapping may include filtering with a plurality of bandpass filters. The BCG signal may be obtained using a remote non-invasive radar detector that includes: at least one radar transmitter (Tx), configured to transmit a THz signal to a predefined body tissue of the subject; at least one radar receiver (Rx), configured to receive a reflected THz signal reflected from the body tissue of the subject; and a radar detector processor, communicatively coupled with the radar transmitter and the radar receiver and configured to process the received reflected THz signal to generate an encoding representative of cardiac related information of the subject. The subject classification may include at least one characteristic of: age, gender, race, a physiological condition, a mental condition, a health condition; and any combination thereof. The model may be generated using a machine learning process of: a neural network algorithm; an artificial neural network algorithm; a convolutional neural network algorithm; a recurrent neural network algorithm; a deep learning algorithm; a linear regression model; a logistic regression model; a data clustering model; a linear classifier model; a support vector machine (SVM) model; a random forest process; and any combination thereof. The method may include simultaneously monitoring and/or identifying multiple subjects in a location.

In accordance with another aspect of the present invention, there is thus provided a system for biometric identification. The system includes a cardiac signal detector, configured to obtain a cardiac signal of at least one reference subject. The system further includes a cardiac signal processor, configured to segment the obtained cardiac signal into a plurality of heartbeat segments over a selected time duration, to apply at least one linear mapping to each of the heartbeat segments to produce a respective heartbeat frequency encoding, and to assign each heartbeat frequency encoding an identification label relating to the reference subject. The system further includes a machine learning processor, configured to apply at least one machine learning process to a collection of heartbeat frequency encodings during a modelling stage, to generate a model for subject classification. The machine learning processor is further configured to apply the model on an input heartbeat frequency encoding during an identification stage, to classify the input heartbeat frequency encoding as belonging to a reference subject if a matching classification is obtained or to determine that the input heartbeat frequency encoding belongs to a non-reference subject if no matching classification is obtained. The cardiac signal may be a ballistocardiograph (BCG) signal. The cardiac signal may be obtained using contactless means. Segmenting the obtained cardiac signal may be performed by applying at least one process of: peak detection; zero-crossing detection; RR interval detection; and/or interbeat (IBI) interval detection. Applying a linear mapping may include filtering with a plurality of bandpass filters. The BCG signal may be obtained using a remote non-invasive radar detector that includes: at least one radar transmitter (Tx), configured to transmit a THz signal to a predefined body tissue of the subject; at least one radar receiver (Rx), configured to receive a reflected THz signal reflected from the body tissue of the subject; and a radar detector processor, communicatively coupled with the radar transmitter and the radar receiver and configured to process the received reflected THz signal to generate an encoding representative of cardiac related information of the subject. The subject classification may include at least one characteristic of: age, gender, race, a physiological condition, a mental condition, a health condition; and any combination thereof. The model may be generated using a machine learning process of: a neural network algorithm; an artificial neural network algorithm; a convolutional neural network algorithm; a recurrent neural network algorithm; a deep learning algorithm; a linear regression model; a logistic regression model; a data clustering model; a linear classifier model; a support vector machine (SVM) model; a random forest process; and any combination thereof. The system may include simultaneously monitoring and/or identifying multiple subjects in a location.

### BRIEF DESCRIPTION OF THE DRAWINGS

The present invention will be understood and appreciated more fully from the following detailed description taken in conjunction with the drawings in which:

FIG. 1 is a schematic illustration of a system for biometric identification, constructed and operative in accordance with an embodiment of the present invention;

FIG. 2 is an illustration of an exemplary cardiac signal segmentation, operative in accordance with an embodiment of the present invention;

FIG. 3 is an illustration of an exemplary frequency encoding of a single heartbeat segment subjected to six different linear filters, operative in accordance with an embodiment of the present invention;

FIG. 4 is a schematic illustration of a flow diagram of a biometric identification method, operative in accordance with an embodiment of the present invention; and

FIG. 5 is a block diagram of a method for biometric identification, operative in accordance with another embodiment of the present invention.

### DETAILED DESCRIPTION OF THE EMBODIMENTS

The present invention overcomes the disadvantages of the prior art by providing a method and system for uniquely identifying a living subject using a distinct physiological signature extracted from cardiac information. The subject is uniquely identified using a single heartbeat obtained from a cardiac signal, such as a BCG signal or a segment of such a signal, which may be obtained in a contactless manner. The disclosed system and method involve segmenting and filtering an obtained cardiac signal to produce a unique frequency encoding representative of the subject heartbeat. The encoding is assigned a corresponding identifying label and fed to a supervised learning process to generate classifications and models of the subject based on different characteristic and profiles, which are then utilized to facilitate their identification. The disclosed system and method allow for real-time identification of persons with a high degree of accuracy and with minimal resources. Identification of the subject may be applied to various uses, such as healthcare monitoring. Furthermore, since cardiac signals can be extracted directly from living skin tissue, the disclosed method may be used to distinguish human skin from other surfaces in video image content

The terms “user” and “operator” are used interchangeably herein to refer to any individual person or group of persons using or operating the method or system of the present invention, such as a person implementing an identification process of a subject to be identified.

The terms “subject” and “living subject” are used interchangeably herein to refer to an individual upon which the method or system of the present invention is operated upon, such as a person to be identified. The subject may be any living entity, such as a person, human or animal, characterized with a functioning heartbeat associated with a cardiac cycle of its heart.

The term “repeatedly” as used herein should be broadly construed to include any one or more of: “continuously”, “periodic repetition” and “non-periodic repetition”, where periodic repetition is characterized by constant length intervals between repetitions and non-periodic repetition is characterized by variable length intervals between repetitions.

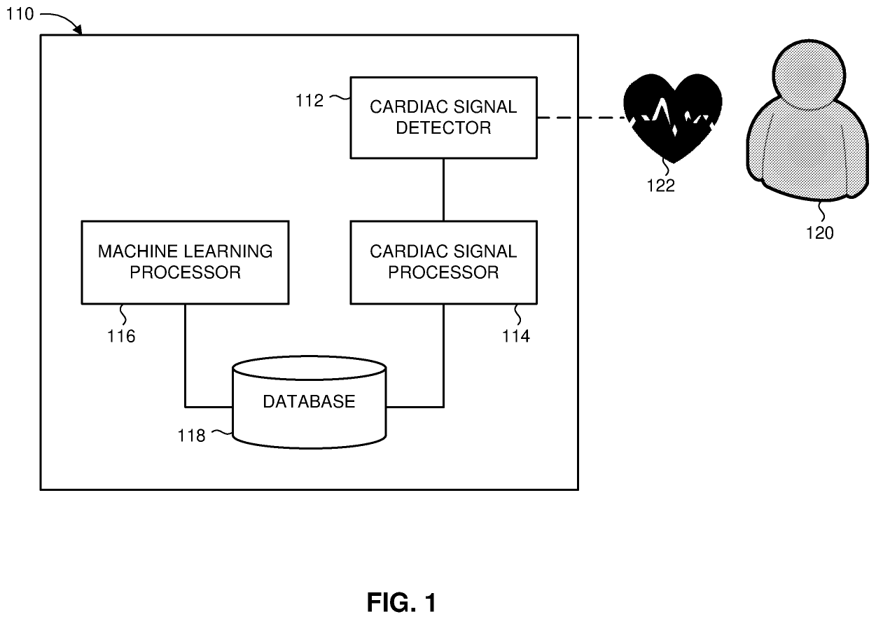

Reference is now made to FIG. 1, which is a schematic illustration of a system, generally referenced 110, for biometric identification, constructed and operative in accordance with an embodiment of the present invention. System 100 includes a cardiac signal detector 112, a cardiac signal processor 114, a machine learning processor 116, and a database 118. Cardiac signal processor 114 is communicatively coupled with cardiac signal detector 112 and with database 118. Machine learning processor 116 is coupled with database 118.

Cardiac signal detector (CSD) 112 is configured to obtain a cardiac signal, referenced 122, relating to a cardiac cycle of a subject, referenced 120. For example, CSD 112 may include one or more ballistocardiogram (BCG) sensors, operative to obtain a BCG signal relating to the vibrations or ballistic forces in the body resulting from cardiac activity. Alternatively, CSD 112 may be an electrocardiogram (ECG) machine, operative to obtain an ECG signal representing electrical activity associated with the cardiac cycle. Further alternatively, CSD 112 may be a photoplethysmogram (PPG) device operative to obtain a PPG signal representing volumetric variations in blood circulation associated with the cardiac cycle, such as a pulse oximeter. Yet further alternatively, CSD 112 may be a contact-based sensor configured to obtain a measurement of body motion associated with blood circulation linked to the cardiac cycle, such as a pressure sensor attached or worn on the body (e.g., a cuff pressure gauge). Accordingly, cardiac signal 122 may represent any applicable signal relating to a cardiac cycle of subject 120, including but not limited to: a BCG signal; an ECG signal; a PPG signal; a body motion signal associated with blood circulation; and the like. CSD 112 may be embodied by a radar detector which transmits a THz radar signal to a body part of subject 120 (e.g., on the chest or the back), and receives and process the reflected THz radar signal to generate a cardiac signal (e.g., a BCG signal), such as described for example in PCT application publication WO2018/167777A1 to Neteera Technologies, entitled “Method and device for non-contact sensing of vital signs and diagnostic signals by electromagnetic waves in the sub terahertz band”, and PCT application publication WO2020/012455A1 to Neteera Technologies, entitled “A sub-THz and THz system for physiological parameters detection and method thereof”. The term “Terahertz (THz)” as used herein refers to Terahertz and sub-Terahertz radiation, corresponding to sub-millimeter and millimeter wave radiation, such as electromagnetic waves within the frequency band between 0.03 to 3 THz, corresponding to radiation wavelengths between 10 mm to 0.1 mm. It is noted that CSD 112 preferably operates in a contactless manner, i.e., without requiring direct physical contact with the subject, such as via an aforementioned radar detector which obtains a BCG signal remotely and does not require a device component being in direct physical contact with subject 120 or being worn or attached to subject 120. Alternatively, CSD 112 may obtain cardiac signal 122 via a contacting measurement, such as ECG electrodes or a pulse oximeter positioned or attached to a body part of subject 120. It is further noted that a radar detector type of CSD 112 may allow for obtaining a cardiac signal from any direction of subject 120, such as from in front or behind or from a non-orthogonal angle relative to subject 120 (i.e., with respect to where the measurement radar signal is transmitted and received). Moreover, a radar detector type of CSD 112 may effectively obtain a cardiac signal in low light or poor visibility conditions, and without necessarily having a direct line of sight to the measured body part of subject 120, such as passing through certain types of obstructions or material barriers (e.g., various kinds of clothing that might be worn by subject 120).

Cardiac signal processor (CSP) 114 and machine learning processor (MLP) 116 receive information or instructions from other components of system 100 and perform required data processing. For example, CSP 114 receives and processes a cardiac signal 122 obtained by CSD 112 to extract a unique identifier therefrom, as will be elaborated upon further hereinbelow. Similarly, MLP 116 analyzes processed cardiac signal information and/or associated identifiers obtained from CSP 114 to generate identification and classification information, as will be elaborated upon further hereinbelow. Database 118 stores relevant information to be retrieved and processed by CSP 114 and/or MLP 116, such as processed cardiac signal data and associated identification and classification information. Database 118 may be represented by one or more local servers or by remote and/or distributed servers, such as in a cloud storage platform.

Information may be conveyed between the components of system 110 over any suitable data communication channel or network, using any type of channel or network model and any data transmission protocol (e.g., wired, wireless, radio, WiFi, Bluetooth, and the like). For example, system 110 may store, manage and/or process data using a cloud computing model, and the components of system 110 may communicate with one another and be remotely monitored or controlled over the Internet, such as via an Internet of Things (IoT) network. The components and devices of system 110 may be based in hardware, software, or combinations thereof. It is appreciated that the functionality associated with each of the devices or components of system 110 may be distributed among multiple devices or components, which may reside at a single location or at multiple locations. For example, the functionality associated with CSP 114 and/or MLP 116 may be distributed between a single processing unit or multiple processing units. CSP 114 and/or MLP 116 may be part of a server or a remote computer system accessible over a communications medium or network, such as a cloud computing platform. CSP 114 and/or MLP 116 may also be integrated with other components of system 110, such as incorporated with CSD 112.

System 110 may optionally include and/or be associated with additional components not shown in FIG. 1, for enabling the implementation of the disclosed subject matter. For example, system 110 may include a user interface (not shown) for allowing a user to control various parameters or settings associated with the components of system 110, a display device (not shown) for visually displaying information relating to the operation of system 110, and/or a camera or imaging device (not shown) for capturing images of the operation of system 110.

The operation of system 100 will now be described in general terms, followed by specific examples. CSD 112 obtains a cardiac signal 122 of a reference subject 120, such as a BCG signal. Cardiac signal 122 may be obtained using a contactless detection technique, such as radar detection. CSP 114 receives and processes the obtained cardiac signal 122. In particular, a continuous cardiac signal is segmented into discrete temporal portions representing individual heartbeats, referred to herein as “heartbeat segments”. The segmentation is performed using any suitable segmentation procedure or algorithm known in the art. For example, the segmentation may be based on: peak detection (i.e., duration between successive peaks of the QRS signal); detection of zero-crossings; detection of RR intervals (i.e., duration between successive R-waves of the QRS signal); detection of interbeat (IBI) intervals; and the like. The processing also includes the application of a linear mapping so as to produce an encoding in the frequency domain. The linear mapping may be performed before or after the segmentation, such that the linear mapping may be applied to each heartbeat segment or to the initial cardiac signal. Accordingly, the signal or heartbeat segments is subject to the linear mapping, such as several linear time invariant filters, e.g., bandpass filters, which may be obtained from a predefined filter bank, to produce a set of filter responses. Alternatively, the linear mapping may involve a Fourier transform, or some type of convolution operator, rather than linear filtering. The linear mapping and segmentation may be combined with an optional non-linear mapping of the values of the segments. For example, non-linear mapping may include: rectification (i.e., obtaining the absolute value of the (complex) signal), followed by exponentiation (e.g., squaring), followed by gain control (i.e., division by the average energy in the signal over a brief time period).

The processing results in several filter responses (or linear mapping responses) corresponding to each heartbeat segment and representing a unique encoding of the subject physiology. The filter responses of a given heartbeat segment may be represented as a matrix of size “hb_length”×“n_filters”, where “hb_length” is the length or duration of the heartbeat segment (i.e., the number of samples), and “n_filters” is the number of filters used to create the filter responses (e.g., number of bandpass/time-invariant filters in the filter bank). The set of filter responses for at least one heartbeat segment is collectively referred to as a “heartbeat frequency encoding”, where the filter responses of a single heartbeat segment represents a minimal encoding of the subject, while filter responses pertaining to a number of successive heartbeat segments of the subject may provide an enhanced subject encoding. Each heartbeat frequency encoding is assigned an identification (ID) label associated with the particular reference subject to whom it belongs. For example, the identification label may include a name, identification number, and/or other personal information relating to the reference subject, e.g., age, gender, location, physical attributes, and the like. The heartbeat frequency encoding and associated ID label is then stored in database 118.

The aforementioned process is repeated for multiple reference subjects to obtain a collection of heartbeat frequency encodings (all of which are stored in database 118). The collected heartbeat frequency encodings are then analyzed by MLP 116 using a machine learning process, to (implicitly) identify different patterns and create models for facilitating the identification and classification of different subjects in accordance with various criteria. The machine learning process may apply machine learning techniques to analyze the training data (i.e., the collected heartbeat frequency encodings) in order to produce mapping functions that can be used for classifying additional instances of new heartbeat frequency encodings according to relevant classification criteria. The data analysis may utilize any suitable machine learning or supervised learning process or algorithm, including but not limited to: an artificial neural network (ANN) process, such as a convolutional neural network, recurrent neural network (RNN), or a deep learning algorithm; a classification or regression analysis, such as a linear regression model; a logistic regression model, or a support-vector machine (SVM) model; a decision tree learning approach, such as a random forest classifier; and/or any combination thereof. The data analysis may utilize any suitable tool or platform, such as publicly available open-source machine learning or supervised learning tools.

MLP 116 establishes classification or profiles of reference subjects whose heartbeat frequency encodings were collected. The classification process may divide the reference subjects into different groups or categories based on common features. For example, the classification process may provide models for identifying subjects in various categories, such as based on: age, gender, location, physical attributes, and the like. Each category may be associated with a relative weighting metric corresponding to a confidence level pertaining to the respective category feature. The different models may then be applied to facilitate the identification of a new subject belonging to the relevant category or classification. The generated models may be iteratively updated and improved based on new information, such as accounting for subsequent successful or unsuccessful subject identifications, and additional heartbeat encodings collected from new reference subjects. Simulations of numerous heartbeat frequency encodings and classifications may also be applied to enhance the reliability and accuracy of the models. The updated models may provide optimal formulas and weighting metrics for different variables or classification features. As more information and statistics are accumulated, the models can be further refined to improve their predictive capability for subject identification.

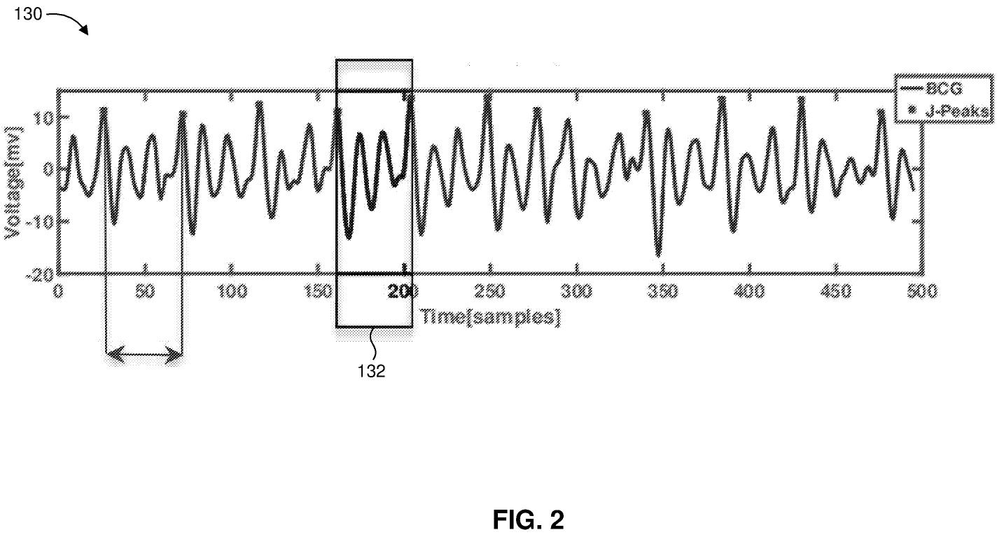

Reference is made to FIG. 2, which is an illustration of an exemplary cardiac signal segmentation, operative in accordance with an embodiment of the present invention. Plot 130 shows a BCG signal (represented as voltage level as a function of time) divided into discrete time segments, such as segment 132, where each segment represents a duration of an individual heartbeat. The segmentation may be performed, for example, using a peak detection algorithm to detect J-peaks of the BCG signal, such that the duration between adjacent maxima J-peaks is designated as an individual heartbeat segment.

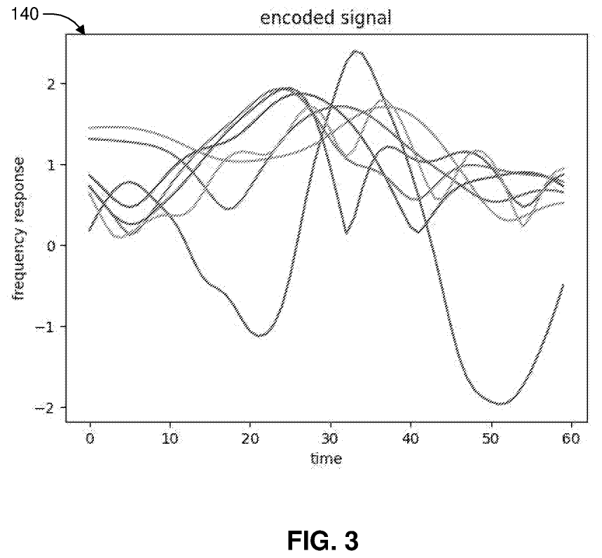

Reference is made to FIG. 3, which is an illustration of an exemplary frequency encoding of a single heartbeat segment subjected to six different linear filters, operative in accordance with an embodiment of the present invention. FIG. 3 shows six different spectral functions, which are plotted in terms of frequency (y-axis) as a function of time (x-axis) in graph 140. The spectral functions are the result of linear filters (and non-linear mappings) applied to a segmented heartbeat signal, where six different filters are applied to produce six different spectral functions with a common start time and end time, and collectively forming a heartbeat frequency encoding. A different number of linear filters may alternatively be applied (to produce a different number of spectral responses). The heartbeat frequency encoding represents an exemplary sample used to train the machine learning process (e.g., neural networks) during the training or modelling phase.

It is appreciated that the system and method of the present invention may provide subject identification from a single heartbeat segment of a cardiac signal, and without requiring devices or components to be in direct physical contact with the subject. Furthermore, the subject does not need to be directly visible to the cardiac signal detector, such as a radar detector, which may operate under poor visibility or light saturation conditions, under obstructions or interference, and from different angles in relation to the subject (e.g., from in front or from behind). Source separation techniques may be utilized to enable measuring and identifying multiple subjects concurrently. The disclosed system does not require costly equipment and has relatively few components, and may be relatively straightforward to operate and maintain. The machine learning analysis may also provide reliable and accurate predictive models, which can be iteratively refined, to enable the identification and classification of new subjects based on the heartbeat frequency encodings.

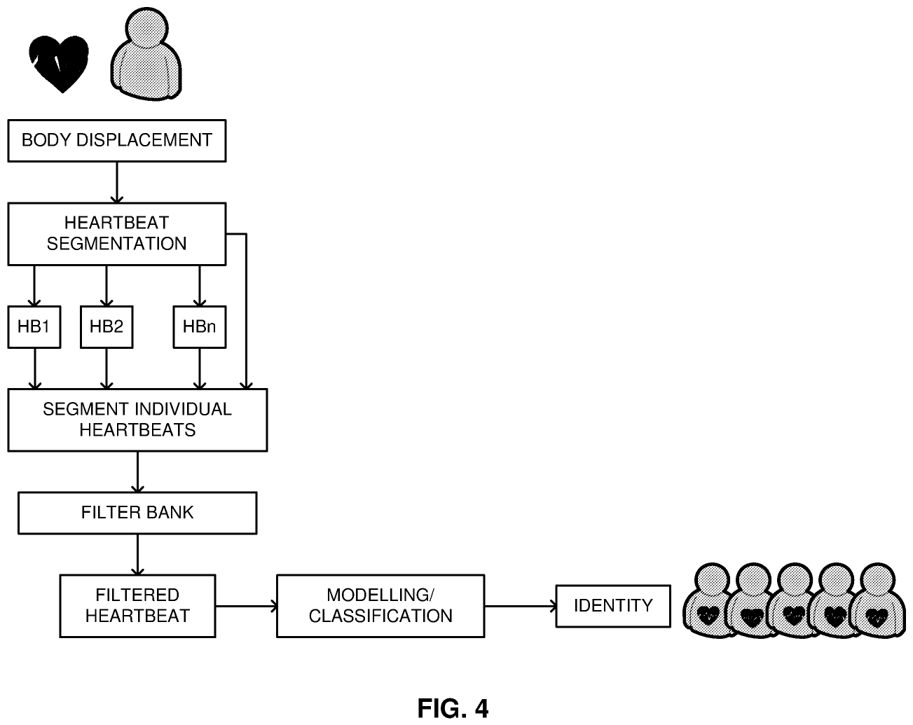

Reference is made to FIG. 4, which is a schematic illustration of a flow diagram of a biometric identification method, operative in accordance with an embodiment of the present invention.

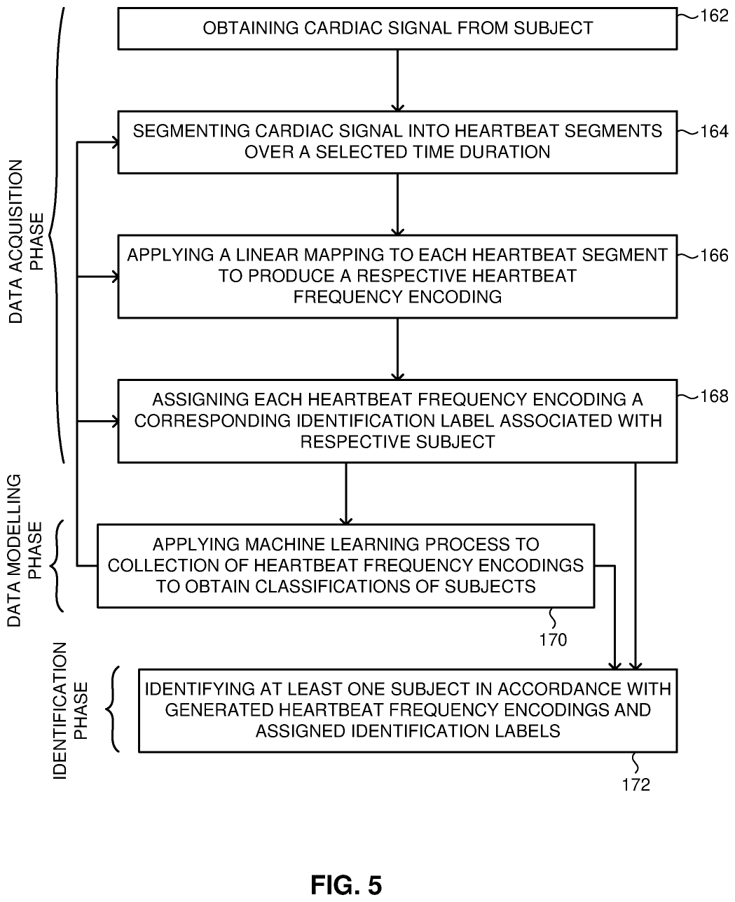

Reference is now made to FIG. 5, which is a block diagram of a method for biometric identification, operative in accordance with an embodiment of the present invention. In procedure 162, a cardiac signal is obtained. Referring to FIG. 1, cardiac signal detector 112 obtains a cardiac signal 122 relating to a cardiac cycle of a reference subject 120, such as a BCG signal. Cardiac signal 122 may be obtained in a contactless manner, and under poor visibility conditions and without direct line of sight to the measurement body region, such as via a designated radar detection unit.

In procedure 164, the cardiac signal is segmented into heartbeat segments over a selected time duration. Referring to FIG. 1, cardiac signal processor 114 receives and processes cardiac signal 122 obtained by cardiac signal detector 112, by performing a segmentation process to divide cardiac signal 122 into individual heartbeats, such as using peak detection, zero-crossing detection, RR interval detection, or interbeat interval detection.

In procedure 166, a linear mapping is applied to each heartbeat segment to produce a respective heartbeat frequency encoding. Referring to FIGS. 1 and 2, cardiac signal processor 114 performs a linear mapping operation, such as by applying a series of bandpass filters or other linear time-invariant filters to cardiac signal 122 or the heartbeat segments thereof. CSP 114 may apply an alternative linear mapping, such as a Fourier transform or convolution operator. CSP 114 may also perform an optional non-linear mapping, such as a rectification operation (obtaining absolute value), exponentiation, and gain control operations. The resulting segmentation and linear mapping processes results in a series of frequency responses for each heartbeat segment representing a unique heartbeat frequency encoding of reference subject 120.

In procedure 168, each heartbeat frequency encoding is assigned a corresponding identification label associated with the respective subject. Referring to FIGS. 1 and 4, cardiac signal processor 114 assigns an ID label for reference subject 120 to the generated heartbeat frequency encoding, including name, ID number and/or additional personal information pertaining to reference subject 120, and stores the information in database 118.

In procedure 170, a machine learning process is applied to a collection of generated heartbeat frequency encodings to obtain a model for classification of subjects. Referring to FIGS. 1 and 4, machine learning processor 116 performs a machine learning process, such as one or more supervised learning techniques known in the art, to the collected heartbeat frequency encodings and associated ID labels, to produce a model. The model is capable, when applied on an input of the same representation such as the inputs it was trained on (i.e., a filtered heartbeat), to produce a classification of the subject as belonging to one of the reference groups of subjects introduced in the training stage or to determine that the sample belongs to a stranger, i.e., a person that does not belong to one of the reference subjects and is thus unknown to the model.

In procedure 172, at least one subject is identified in accordance with the generated heartbeat frequency response encodings and assigned identification labels. Referring to FIGS. 1 and 4, machine learning processor 116 performs a machine learning process to identify a new subject, in accordance with the profiles and classification generated during the modeling phase. Source separation techniques known in the art may be applied to enable monitoring and identifying multiple subjects in a location simultaneously.

The method of FIG. 5 is generally implemented in an iterative manner, such that at least some of the procedures are performed repeatedly, in order to provide for a dynamic biometric identification of multiple subjects in real-time.

The disclosed biometric identification method can be used for various applications. For example, the disclosed identification method can be used to associate collected BCG (or other cardiac signal) data with a general health information file of the identified subject, which may reside in cloud storage. The disclosed identification method may also be used to monitor vulnerable or incapacitated individuals, such as to identify if a person in an eldercare home or assisted living facility has inadvertently entered a room or used a personal item belonging to somebody else (e.g., has laid down on someone else's bed), and to notify a staff member accordingly.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A method for biometric identification, the method comprising the procedures of:
obtaining a cardiac signal from at least one reference subject;
segmenting the obtained cardiac signal into a plurality of heartbeat segments over a selected time duration;
applying at least one linear mapping to each of the heartbeat segments to produce a respective heartbeat frequency encoding;
assigning each heartbeat frequency encoding an identification label relating to the reference subject;
applying at least one machine learning process to a collection of heartbeat frequency encodings during a modelling stage, to generate a model for subject classification; and
applying the model on an input heartbeat frequency encoding during an identification stage, to classify the input heartbeat frequency encoding as belonging to a reference subject if a matching classification is obtained or to determine that the input heartbeat frequency encoding belongs to a non-reference subject if no matching classification is obtained.

2. The method of claim 1, wherein the cardiac signal is a ballistocardiograph (BCG) signal.

3. The method of claim 1, wherein the cardiac signal is obtained using contactless means.

4. The method of claim 1, wherein the procedure of segmenting the obtained cardiac signal is performed by applying at least one process selected from the group consisting of:
peak detection;
zero-crossing detection;
RR interval detection; and
interbeat (IBI) interval detection.

5. The method of claim 1, wherein the procedure of applying a linear mapping comprises filtering with a plurality of bandpass filters.

6. The method of claim 1, wherein the BCG signal is obtained using a remote non-invasive radar detector comprising:
at least one radar transmitter, configured to transmit a THz signal to a predefined body tissue of the subject;
at least one radar receiver, configured to receive a reflected THz signal reflected from the body tissue of the subject; and
a radar detector processor, communicatively coupled with the radar transmitter and the radar receiver and configured to process the received reflected THz signal to generate an encoding representative of cardiac related information of the subject.

7. The method of claim 1, wherein the subject classification comprises at least one characteristic selected from the group consisting of:
age; gender; race; a physiological condition; a mental condition; a health condition; and any combination thereof.

8. The method of claim 1, wherein the model is generated using a machine learning process selected from the group consisting of:
a neural network algorithm; an artificial neural network algorithm; a convolutional neural network algorithm; a recurrent neural network algorithm; a deep learning algorithm; a linear regression model; a logistic regression model; a data clustering model; a linear classifier model; a support vector machine (SVM) model; a random forest process; and any combination thereof.

9. The method of claim 1, comprising simultaneously monitoring or identifying multiple subjects in a location.

10. A system for biometric identification, the system comprising:
a cardiac signal detector, configured to obtain a cardiac signal of at least one reference subject;
a cardiac signal processor, configured to segment the obtained cardiac signal into a plurality of heartbeat segments over a selected time duration, to apply at least one linear mapping to each of the heartbeat segments to produce a respective heartbeat frequency encoding, and to assign each heartbeat frequency encoding an identification label relating to the reference subject; and
a machine learning processor, configured to apply at least one machine learning process to a collection of heartbeat frequency encodings during a modelling stage, to generate a model for subject classification, and to apply the model on an input heartbeat frequency encoding during an identification stage, to classify the input heartbeat frequency encoding as belonging to a reference subject if a matching classification is obtained or to determine that the input heartbeat frequency encoding belongs to a non-reference subject if no matching classification is obtained.

11. The system of claim 10, wherein the cardiac signal is a ballistocardiograph (BCG) signal.

12. The system of claim 10, wherein the cardiac signal is obtained using contactless means.

13. The system of claim 10, wherein the cardiac signal processor is configured to segment the obtained cardiac signal by applying at least one process selected from the group consisting of:
peak detection;
zero-crossing detection;
RR interval detection; and
interbeat (IBI) interval detection.

14. The system of claim 10, wherein the linear mapping comprises filtering with a plurality of bandpass filters.

15. The system of claim 10, wherein the BCG signal is obtained using a remote non-invasive radar detector comprising:
at least one radar transmitter, configured to transmit a THz signal to a predefined body tissue of the subject;
at least one radar receiver, configured to receive a reflected THz signal reflected from the body tissue of the subject; and
a radar detector processor, communicatively coupled with the radar transmitter and the radar receiver and configured to process the received reflected THz signal to generate an encoding representative of cardiac related information of the subject.

16. The system of claim 10, wherein the subject classification comprises at least one characteristic selected from the group consisting of:
age; gender; race; a physiological condition; a mental condition; a health condition; and any combination thereof.

17. The system of claim 10, wherein the model is generated using a machine learning process selected from the group consisting of:
a neural network algorithm; an artificial neural network algorithm; a convolutional neural network algorithm; a recurrent neural network algorithm; a deep learning algorithm; a linear regression model; a logistic regression model; a data clustering model; a linear classifier model; a support vector machine (SVM) model; a random forest process; and any combination thereof.

18. The system of claim 10, comprising simultaneously monitoring or identifying multiple subjects in a location.
